(19) United States
(12) Patent Application Publication
(10) Pub. No.: US 2024/0319098 A1
(43) Pub. Date: Sep. 26, 2024

(54) METHOD, APPARATUS AND SYSTEM OF INTERFERING-AGENT COMPATIBLE BIOMOLECULE STORAGE, TRANSPORT AND QUANTIFICATION

(71) Applicant: ProtiFi, LLC, Fairport, NY (US)

(72) Inventor: John Paul Wilson, Fairport, NY (US)

(21) Appl. No.: 18/579,042

(22) PCT Filed: Jul. 13, 2022
(86) PCT No.: PCT/US22/37021

§ 371 Date: Jan. 12, 2024

Related U.S. Application Data
(60) Provisional application No. 63/203,221, filed on Jul. 13, 2021.

(51) Int. Cl. G01N 21/64; C12Q 1/6806; G01N 1/34; G01N 30/88; G01N 33/68

(52) U.S. Cl. CPC G01N 21/6486; C12Q 1/6806; G01N 1/34; G01N 30/88; G01N 33/6827; G01N 2030/8827

(57) ABSTRACT

A method of quantifying target molecules comprising the steps of: binding target molecules to a surface, wherein the target molecules are presented for a quantification assay; cleaning the target molecules of contaminating reagents, wherein the target molecules remain bound to the surface; directly quantifying the target molecules, wherein the target molecules remain bound to the surface, wherein direct quantification of the target molecules is performed by measurement of intrinsic fluorescence of the target molecules.

This application is a national stage of International Application No. PCT/US22/37021, filed Jul. 13, 2022, which claims priority of U.S. Provisional Patent Application Ser. No. 63/203,221, filed on Jul. 13, 2021, the contents of which are expressly incorporated herein by reference herein.

### FIELD

The present invention relates to a method, apparatus and/or system of sample storage and quantification which is compatible with the presence of agents that can interfere with sample stability and/or the quantification of the molecules of interest.

### BACKGROUND

In the arts of the analysis of biomolecules including and not limited to biochemistry, protein sequencing, molecular biology, genomics, proteomics and other related fields, among many others, there exists the challenges of first quantifying the class of biomolecule under study—micrograms of protein, DNA or RNA, by example—and ascertaining the identity of the biomolecule(s) under study. Owing to their linear nature, the identity of proteins, peptides, RNA, DNA and related molecules is defined by their sequence. Many technologies have been developed for the sequencing of linear biomolecules: the mainstays of proteomics and genomics.

Research and study of biological samples contains many steps. Samples must first be collected, are then typically stored and/or transported, are treated and/or prepared through a variety of chemical, physical, biochemical and other processes such as chromatography, and are then able to be analyzed via a manifold variety of techniques: arrays, aptamer or antibody or surface binding (each readable through a wide variety of techniques), mass spectrometry, etc. Recent advances in analytical throughput, in some cases now requiring only minutes or even second per sample to identify and quantify the molecules under study, necessitate concomitant advances in sample handling and preparation. Specifically, the simplification and ideally elimination of any and all steps in the workflows of sample storage, transport, preparation and analysis is desirable to increase both throughput and robustness.

Due to their liability, the storage and shipment of samples, especially those of biological origin, is frequently performed at cold temperatures: −20 C, −80 C or even in liquid nitrogen. This strategy is first expensive, second easily subject to failure, for example during power outages, and third not necessarily easily accessible: freezers are big and difficult to move and power, and ice, dry ice, liquid nitrogen or other coolants run out and are difficult and expensive to ship. Indeed in drug trials, sample shipment costs can constitute 50% of the overall cost. Thus, significant improvements in the ability to store and ship samples in a more efficient, inexpensive manner, especially if possible at room temperature, are necessary.

### SUMMARY

One aspect of the present application relates to a method of quantifying target molecules comprising the steps of: binding target molecules to a surface, wherein the target molecules are presented for a quantification assay; cleaning the target molecules of contaminating reagents, wherein the target molecules remain bound to the surface; directly quantifying the target molecules, wherein the target molecules remain bound to the surface, wherein direct quantification of the target molecules is performed by measurement of intrinsic fluorescence of the target molecules.

In certain embodiments, further comprising storing or transporting the target molecules at least at room temperature, the target molecules remain stable while bound to the surface. In certain embodiments, the target molecules have a greater affinity for the surface than the affinity for the surface exhibited by the contaminating reagents. In certain embodiments, the direct quantification occurs by use of spectrophotometric techniques. In certain embodiments, each step of the method is automated. In certain embodiments, the surface is a C18 hydrophobic surface or optionally C4, C8, or other suitable hydrophobic surface. In certain embodiments, the target molecules are bound to a surface by hydrophobic or hydrophilic chromatography. In certain embodiments, the target molecules are bound to a surface by weak or strong ion exchange (cation or anion). In certain embodiments, the target molecules are bound on a surface presented on one or more selected from the group consisting of beads, membrane, packed column, monolithic column, glass beads and chromatographic beads. In certain embodiments, the target molecules are bound on the surface and washed of reducing reagents. In certain embodiments, the target molecules are bound on the surface and washed of aniline. In certain embodiments, the direct quantification is performed using a bicinchoninic acid assay. In certain embodiments, the direct quantification is performed by measuring protein fluorescence. In certain embodiments, the target molecules are nucleic acids. In certain embodiments, the target molecules are RNA. In certain embodiments, the target molecules are proteins. In certain embodiments, the intrinsic fluorescence of tryptophan is measured.

Another aspect of the application is an apparatus for implementation of the methods and systems described herein, wherein the device comprises: a protein immobilization spot; a UV light source; and a detector. In certain embodiments, the apparatus comprises a 96-well plate. In certain embodiments, the apparatus is automated.

Another aspects of the application is the storage or transportation of target molecules in conjunction with the methods described herein.

### BRIEF DESCRIPTION OF THE DRAWINGS

While the present disclosure will now be described in detail, and it is done so in connection with the illustrative embodiments, it is not limited by the particular embodiments illustrated in the figures and the appended claims.

FIG. 1 shows three amino acid residues that are primarily responsible for the inherent fluorescence of proteins.

FIG. 2 shows an S-trap column for protein capture.

FIG. 3 shows results were obtained with excitation at 277 and emission at 360 nm.

FIG. 4 shows BSA response (277/350) in solution

FIG. 5 shows BSA Response (277/350) in S-trap with digestion buffer.

FIG. 6 shows BCA assay with BSA samples.

FIG. 7 shows BSA Response (277/410) in digestion buffer.

FIG. 8 shows room temperature stability of samples.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

Steps of sample preparation are necessary because samples, especially those of biological origin, are typically complex and contain many different molecular classes, each of which may necessitate its own treatment, Additionally, the presence of certain molecules can disturb both the ability to quantify (and/or analyze) the molecules of interest or also disturb the stability of the sample. For example, the steps and techniques to analyze lipids are different from those to analyze proteins, and the presence of a large quantity of lipid can easily interference with the analysis of proteins. Similarly, we seek to avoid the presence of enzymes or other molecules which modify or damage the molecules of interest, By example, RNAses must be inactivated or eliminated for successful transcriptomics analyses, and the presence of undesired proteases can hinder or prevent the study of proteins. These are solely illustrative examples and do not serve to limit the field of the invention.

Quantification of a particular class or classes of molecule, such as protein, RNA, DNA, lipid, glycan or metabolite concentration, is a common and often obligatory step in omics analyses such as proteomics, transcriptomics, (to a degree) genomics, lipidomics, glycomics and metabolomics (collectively, “omics,” which does not exclude other kinds of analyses): samples such as blood, lysates (of e.g. cells or tissue), urine etc. have varying concentrations, and we often desire to analyze the same quantity of the molecule class of interest. For example, in proteomics, researches often seek to normalize or set equal the amount of protein that is analyzed in each sample. Proteins, or other molecular classes, also might be used as a surrogate to approximate the concentration of other molecular classes such as in metabolomics or other kinds of omics studies, By an example that is not limiting, one might use the protein concentration of a body fluid like urine or serum as a surrogate to estimate the overall concentration of other molecular classes.

One skilled in the art will recognize that in general, chromatography, thin layer chromatography (TLC), gas chromatography (GC), and high pressure liquid chromatography (HPLC), HPLC and C with or without mass spectrometric detection, as well as Surface Plasmon Resonance (SPR) can all be used to identify and quantify molecules of multiple classes. In mass spectrometry, stable isotope techniques, as well as isobaric techniques, are well established and deployed for quantification. Similarly, measurement of bulk physical properties like density, electrical conductivity and ultrasonic velocity can be used to quantify the amount of molecule present in a sample. Likewise, measurements of adsorption of radiation including the absorbance of and/or fluorescence with ultraviolet-visible radiation, infrared radiation (including FTIR), X-ray radiation or Nuclear Magnetic Resonance (NMR) are routinely used to determine concentrations and molecular identities, Measurement of scattering of radiation can also be used such as light or ultrasonic scattering via turbidity or ultrasonic velocity and absorption of ultrasound can also be used. The exact parameters of the use of each of these techniques depends on the properties of the molecule of interest, which is known to one skilled in the art.

Quantification techniques to determine the concentration of biomolecules and other molecules are well established and fall into two broad classes:

Assays that determine the total amount of some type or class of molecule, such as total protein assays, total DNA assays, total RNA assays, total lipid assays, total glycosylation assays or total metabolite assays; and

Those which are specific to one molecule or family of molecule. These assays are often based on specific affinity via antibodies, lectins, specific sequences of DNA or RNA, aptamers, etc., and they can be to broad classes such as with antibodies raised to a conserved region of homology in a family of proteins or enzymes. These specific assays may also be based on a particular activity of a molecule, such as an enzymatic action.

Both classes of assays are well known to one in the art. For example, to determine RNA or DNA concentration (or protein), one often uses UV spectroscopy wherein the absorbance of a sample is measured at or around 260 nm (representative of nucleic acids) and at or around 280 nm (representative of proteins; a blank might be measured at a higher wavelength like 320 nm) and the nucleic acid concentration, or protein concentration, is calculated using the Beer-Lambert law. An A260 reading of 1.0 is equivalent to ˜40 g/ml single-stranded RNA and the A260/A280 ratio is used to assess RNA purity; an A260/A280 ratio of 1.8 2.1 is indicative of highly purified RNA, Alternatively, fluorescence can be employed wherein nucleic acids can be excited at or around 260-270 nm and emission around the 300-400 nm range, which one skilled in the art recognizes is dependent on a large number of factors such as sequence and chemical environment including pH, ionic concentration, other ions and quenchers present, etc. It is of particular importance to note that many, many molecules both absorb and fluoresce, and their concomitant (contaminating) presence along with the molecules of interest, of any class, leads to incorrect quantifications.

Nucleic acids may also be quantified with the help of fluorescent dyes that bind to DNA and/or RNA, As with other assays, samples and a series of standards of known concentrations for back calculation of the unknown concentrations are assayed. Samples are incubated with a dye that binds to the nucleic acid and undergoes a conformational change, resulting in increased fluorescence at a wavelength specific to the dye being used. Fluorescence is measured, and a standard curve (for plate readers) or reference standard (for handheld fluorometers) is created by plotting fluorescence against nucleic acid concentrations of the known standards. The fluorescence of the unknown sample then is converted to a nucleic acid concentration using the linear regression equation that best describes the standard curve. The main advantage to using fluorescent dye-based methods for RNA quantification is sensitivity; dyes which absorb but do not fluoresce can also be used. Examples of dyes include ethidium bromide, propidium iodide, crystal violet, DAPI (4′,6-diamidino-2-phenylindole), 7-AAD (7-aminoactinomycin D), Hoechst 33258 (33342, 34580) and YOYO-1/DiYO-1/TOTO-1/DiTO-1 are examples of dyes that offer different colors of fluorescence (both excitation and emission) or absorbance, membrane permeability, toxicity, sensitivity, as well as other properties.

Nucleic acid analysis is automated in an Agilent 2100 Bioanalyzer, which uses dyes and microfluidics and sample-specific chips for analysis. This system is essentially a miniaturized version of agarose and acrylamide gels used to separate nucleic acid and proteins for analysis. Samples are combined with a fluorescent dye and injected into wells in the chip. The samples move through a gel matrix in the microchannels and are separated by electrophoresis. The samples then are detected by fluorescence, and electropherograms and gel-like images are created by the data analysis software for sizing and quantification

One skilled in the art will knows that an assay for a specific sequence of nucleic acid, DNA or RNA, can be done via amplification such as polymerase chain reaction (PCR), strand displacement assay (SDA), transcription-mediated assay (TMA) or ligase chain reaction, among other possibilities. Subsequently, microarrays, which quantify a set of predetermined sequences, and RNA-Seq, which uses high-throughput sequencing to record all transcripts, can be used to quantify the specific nucleic acids. Readout is via a multitude of analytical platforms including 454 Life Sciences, Illumina, SOLiD, Ion Torrent or PacBio, among others. These workflows and techniques are well established, widespread and commercially available.

Lipids might be assayed with the sulfo-phospho-vanillin [SPV] assay for a simple colorimetric readout; however, the sensitivity of this assay can be limiting and it requires a significant amount of sample. The chemical reactions are complex and are thought to involve formation of relatively stable (up to several hours) carbonium ion (or carbocation) chromogen in the initial reaction followed by generation of a pink chromophore upon addition of vanillin to the reaction. Alternatively infrared (IR) spectroscopy, mass spectrometry (MS) or NMR may be used to assay lipid amount or concentration. In NMR lipid content is determined by measuring the area under a peak in an NMR chemical shift spectra that corresponds to the lipid fraction. Lipids, as well as other molecular classes, may also be assayed via differential solubility and gravimetric means. For example, an aqueous sample might be shaken with hexane, and the hexane removed into a tarred container, Upon evaporation lipids will be both measurable by their weight and isolated from the other molecular classes. The Babcock, Gerber or detergent methods, familiar to one skilled in the art, may also be used.

Glycans can be quantified with lectins, including lectin arrays and blots, as well as through chromatographic methods, which also allow for the concentration of specific glycans to be determined by calculating the peak area one can assess the percentage of a specific type of oligosaccharide out of the total glycan repertoire. Lectins which are marked or labeled via fluorophores or chromophores or linked enzymes (e.g. HRP) or heavy isotopes (etc.) may also be used, One skilled in the art will know that glycans can be detected, identified and quantified mass spec and chromatographic techniques. Other techniques for glycan quantification include labeling reagents such as RapiFluor-MS, 2-aminobenzamide or procainamide among others, S_{O} labeled glycans are detected via HPLC, often using hydrophilic interaction chromatography (HILIC), with fluorescence detection. Quantification may also be determined via a permethylation using isotopic labeling: glycans are labeled with either 12C- or 13C-methyl iodide wherein samples are compared or one sample is a standard and the labeled glycans are analyzed by MS. This method has a high-dynamic range, adequate linearity, and high reproducibility. Colorimetric assays are also known: simple sugars, oligosaccharides, polysaccharides, and their derivatives, including the methyl ethers with free or potentially free reducing groups, give an orange yellow color when treated with phenol and concentrated sulfuric acid. The reaction is sensitive and the color is stable. Multiple other reagents and reactions such as p-anisidine hydrochloride are also known; see Timell, T. E., C. P. J. Glaudemans, and A. L. Currie. Spectrophotometric methods for determination of sugars. Analytical chemistry 28.12 (1956): 1916-1920, Chromatography can again be used for the determination of the composition of labeled or derivatized glycans, polysaccharides and/or their methyl derivatives.

Techniques for the determination of protein amount and protein concentration are very well established and include fluorometric or colorometric protein assays such as the Biuret reaction, Lowry method, Coomassie Blue (CB) G-250 dye-binding assay (Bradford assay) and bicinchoninic acid (BCA) assay, among others such as UV absorbance or fluorescence strategies and fluorogenic reagents that fluoresce after reaction such as with fluorescamine and 2-methoxy-2,4-diphenyl-3(2H)-furanone (MDPF), among others. Many techniques, familiar to one skilled in the art, exist for the quantification of specific proteins via ELISA, aptamer, antibody, array and other affinity-based approaches in addition to activity based (such as enzyme activity), as well MS-based quantification, Olink and Somalogic strategies, etc.

An overview of protein assays is found at thermofisher.com and archived at the Internet Archive Wayback Machine as of Jul. 13, 2022. An overview of concentration assays for nucleic acids is found at promega.com and archived at the Internet Archive Wayback Machine as of Jul. 13, 2022.

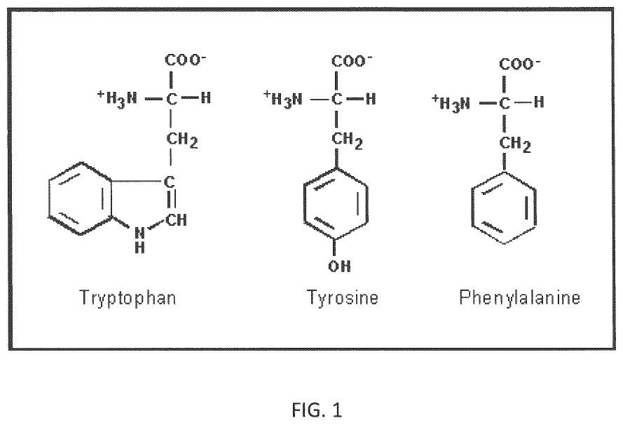

Proteins have intrinsic fluorescence via three amino acid residues that are primarily responsible for the inherent fluorescence of proteins are tryptophan, tyrosine and phenylalanine (FIG. 1). These residues have distinct absorption and emission wavelengths and differ in the quantum yields (Table 1 below). The intrinsic fluorescence of proteins can consequently be used to determine protein concentration.

Tryptophan is much more fluorescent than either tyrosine or phenylalanine. However, the fluorescent properties of tryptophan are solvent dependent. As the polarity of the solvent decreases, the spectrum shifts to shorter wavelengths and increases in intensity. For this reason, tryptophan residues buried in hydrophobic domains of folded proteins exhibit a spectral shift of 10 to 20 nm. This phenomenon has been utilized to study protein denaturation; see Principles of Fluorescence Spectroscopy 2nd Edition (1999) Lakowicz, JR. Editor, Kluwer Academic/Plenum Publishers, New York, New York. Tryptophan typically has a wavelength of maximum absorption of 280 nm and an emission peak that is solvatochromic, ranging from ca. 300-350 nm, depending on the polarity of the local environment (see, e.g., Intrinsic Fluorescence of Proteins and Peptides at dwb.unl.edu, incorporated herein by reference in its entirety for all purposes.

Tyrosine can be excited at wavelengths similar to that of tryptophan, but emits at a distinctly different wavelength. While tyrosine is less fluorescent than tryptophan, it can provide significant signal, as it is often present in large numbers in many proteins. Tyrosine fluorescence has been observed to be quenched by the presence of nearby tryptophan moieties via resonance energy transfer, as well as by ionization of its aromatic hydroxyl group.

Phenylalanine is very weakly fluorescent and can only be observed in the absence of both tryptophan and tyrosine. Due to tryptophan's greater absorptivity, higher quantum yield, and resonance energy transfer, the fluorescence spectrum of a protein containing the three amino acids usually resembles that of tryptophan.

TABLE 1

| Absorption | | Fluorescence | | |
|---|---|---|---|---|
| | Wavelength | | Wavelength | Quantum |
| Amino Acid | (nm) | Absorptivity | (nm) | Yield |
| Tryptophan | 280 | 5,600 | 348 | 0.20 |
| Tyrosine | 274 | 1,400 | 303 | 0.14 |
| Phenylalanine | 257 | 200 | 282 | 0.04 |

All quantification and identification techniques have their own limitations and tolerances. For example, both protein and nucleic acids can be quantified by their absorbance at 280 nm and 260 nm respectfully; see the original at Warburg, O. and W. Christian (1942) Biochem. Z. 310:384-421. However, many other molecules buffers, detergents and dyes or pigments, just to name a few—also absorb in this region. Similarly, chemical assays such as a BCA or Bradford assay for protein quantification are subject to various interferences specific to their chemistry. BCA, by example, cannot be effectively used in a reductive solution and Bradford cannot be effectively used in a detergent containing solution, (The concentration of the interfering agents may be low enough that it can be accounted for through the inclusion of a standard curve containing the same interfering reagents, as is known to one skilled in the art.) Likewise, many molecules fluoresce and yield false positive signals if they are present. While these are limitations of two types of protein assays, it is not a limiting example and one skilled in the art will recognize the types of contamination(s) that can lead to false readings in either direction of concentration or amount.

Finally, assays typically sacrifice a portion of sample, add cost and time and are subject to interferences (reducing reagents or surfactants, respectfully, by example for the last two assays) as well as edge effects in 96-well plates, Indeed, for small samples such as in single-cell studies, or samples from laser microcapture dissection, the sample quantity is too little for almost any assay. Thus, obviating the need for additional protein assays, or other assays for different (bio)molecular classes, is desirable to increase the efficiency of proteomics and other omics fields.

### Method

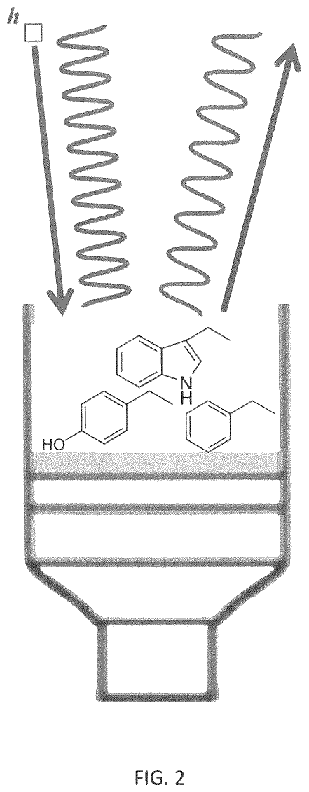

This invention flows from the surprising discovery that molecules can be bound to a surface, cleaned of contaminating reagents then directly quantified directly, in place on that surface, using the intrinsic fluorescence of the molecule of interest (FIG. 2). This result was especially surprising as one skilled in the art would typically expect that surface-bound molecules would be quenched, which was not the case. This invention enables the use of reagents during sample preparation that would otherwise interfere with later protein assays, such as reducing agents in the solubilization of keratin or FFPE samples. This invention is a general-purpose technique that, for protein analyses, makes the entire range of lysis buffer reagents compatible with protein quantification; it extends similar flexibility to other omics kinds. In embodiments using intrinsic fluorescence, this invention also allows for the quantification of very small quantities of sample that would otherwise be completely used up in assays of molecular content.

The invention further flows from the surprising discovery that such samples, once surface bound, were amazingly found to be stable for months at least at room temperature, and can be stored and shipped without cooling or special accommodations, as described in the examples below.

As with all solid-phase chromatography, sample applied to S-Trap columns loads first at the head of the column or well (FIG. 2). As sites of affinity are occupied, the band of column loading progresses deeper into the protein trap. This surface-concentrated presentation of intrinsically fluorescent tryptophan, tyrosine and phenylalanine residues allows fluorescent protein quantification via top excitation and top emission detection. Protein quantification occurs in the exact same plate used in downstream sample processing, removing the need for a separate protein assay and the sacrifice of sample for that assay. One skilled in the art will recognize that while protein fluorophores are described herein and while this example describes proteins, this example is not limiting and any molecule which can be physically immobilized, cleaned (without releasing the molecule of interest) of contaminants and displayed can be so analyzed. Key to this invention is that first that the molecules of interest have affinity for a solid support that is different from the contaminants, and second that the so immobilized molecules of interest are presented for an assay, by example by (and not limited to) spectrophotometric techniques.

Further, while intrinsic fluorescence is extremely useful because it is convenient (here, the sample is literally in exactly the same place as typical S-Trap sample processing (see, U.S. Pat. No. 11,009,510 incorporated herein by reference); no additional step was added except to measure protein concentration via fluorescence), non-destructive, and the variable parameters are simply the wavelengths of excitation and emission (or absorption, if molecules are immobilized using something clear, or if the molecules themselves have color, all settings which are easily changed on the appropriate plate reader), any assay appropriate for the molecule kind under study can be deployed. Continuing with the protein assay example above, a protein solution containing both reductants and surfactants applied to the appropriate capture mechanism which allows for washing could then be assayed via either BCA or Bradford assays, or Lowry or fluorescent dye-based assays and fluorogenic amine derivatizations (or derivatizations on other groups), etc.

One skilled in the art will immediately recognize that any form of physical capture compatible with later washing is possible. For example, a C18 hydrophobic surface, or other hydrophobic or hydrophilic chromatography, or weak or strong ion exchange (cation or anion) (beads, membrane, packed column, monolithic column, etc.), glass beads or chromatographic beads, etc. can capture and immobilize the molecules of interest such that they can be washed of contaminants, such as reducing reagents (in the case of a BCA assay) or aniline in the case of protein fluorescence.

One skilled in the art will recognize there are multiple ways to first affix or immobilize the (bio)molecule(s) under study to a solid phase or surface or bead or particle, to wash away the contaminants in a way that leaves the desired molecule, and then to proceed with analyses of quantity and identity. Such attachments may be non-covalent via adsorption or affinity or chromatographic means; see Yeo D S, Panicker R C, Tan L P, Yao S Q. Strategies for immobilization of biomolecules in a microarray. Combinatorial chemistry & high throughput screening, 2004 May 1; 7(3):213-21. Biomolecule chromatography, which will be recognized by one skilled in the art as a means to affix or immobilize biomolecules, is reviewed in Chapter 14 Chromatography of Biomolecules, by Susan R. Mikkelsen, Eduardo Cortón in Bioanalytical Chemistry, John Wiley Interscience, 2004 cf. Mikkelsen S R, Cortón E, Bioanalytical chemistry. John Wiley & Sons; 2016 Mar. 7. Alternatively, covalent chemistries well known to one skilled in the art can be employed to affix or immobilize biomolecules. Reviews and overviews include, among many, many others:
Vashist S K, Luong J R. Antibody immobilization and surface functionalization chemistries for immunodiagnostics. In Handbook of Immunoassay Technologies 2018 Jan. 1 (pp. 19-46). Academic Press.
Grainger D W, Greef C H, Gong P, Lochhead M J Current microarray surface chemistries. In Microarrays 2007 (pp. 37-57). Humana Press.
Todt S, Blohm D H. Immobilization chemistries. DNA Microarrays for Biomedical Research, 2009:81-100.
Sonawane M D, Nimse S B. Surface modification chemistries of materials used in diagnostic platforms with biomolecules. Journal of Chemistry. 2016 Jan. 1; 2016.
Zhu H, Snyder M. Protein chip technology. Current opinion in chemical biology. 2003 Feb. 1; 7(1):55-63,
Oh S J, Hong B J, Choi K Y, Park J W, Surface modification for DNA and protein microarrays, OTICS: A journal of Integrative Biology. 2006 Sep. 1; 10(3):327-43.
Wong L S, Khan F, Micklefield J. Selective covalent protein immobilization: strategies and applications. Chemical reviews. 2009 Sep. 9; 109(9):4025-53.

In one example, we demonstrate this new invention of direct quantification of cleaned, surface-bound protein on S-Trap 9&-well plates using intrinsic protein fluorescence. S-Trap plates and columns are specifically designed to trap protein and clean them of contaminants, such as buffers, reducing agents, detergents and other small molecules. As detailed above these small molecules frequently interfere with protein assays: detergents for Bradford, reducing agents for BCA, anything that absorbs at 280 nm for absorption, and anything fluorescent for quantification by fluorescence.

One skilled in the art will quickly recognize that while the examples herein described used a commercial plate reader, embodiments of this invention include devices, apparatuses and systems specific to the task of the invention. By example, one skilled in the art will quickly understand the utility of automating and/or combining molecular capture, cleaning and quantification, wherein the device contains a target or place of sample immobilization, a means to clean said sample of contaminants, a means to quantify the molecules of interest and a means to measure the signal from said quantification. By non-limiting example, such a device intended for protein analysis using fluorescence excitation and emission contains a protein immobilization spot, potentially automated means of washing said proteins (though this could also be done offline), a UV light source and a detector. Ideally, said device is automated such that multiple samples can be analyzed either in parallel (like a plate reader) or serially (like an autosampler). As described above other assay kinds can be used including SPR.

One skilled in the art will recognize that a 96-well plate is only one of a myriad of possible forms, which is essential because this invention works from large scales down to single molecules, assuming sufficient sensitivity.

The present disclosure is further illustrated by the following examples that should not be construed as limiting. The contents of all references, patents, and published patent applications cited throughout this application, as well as the Figures and Fables, are incorporated herein by reference.

### EXAMPLES

### Example 1

S-Trap 96-well plates were made and used as per standard protocols. Samples were bound onto plates and washed as per standard protocols; standard BSA curves were also loaded in the same way (below).

From 50, 100, 200 and 300 ug of human serum in four aliquots, protein concentration determined by BCA, was diluted into 46 uL of 5% SDS containing 50 mM TEAB. This solution was reduced for 10 min at 55 C with 2 uL of 120 mM added, alkylated with 2 uL of 500 mM MMTS in isopropanol alcohol, then 5 uL of 12% phosphoric acid was added to denature the proteins followed by 350 uL of 100 mM TEAB (final) in 90% MeOH binding buffer. The protein was washed three times with 350 uL of binding buffer. 100 uL of 50 mM TEAB was then added (after measurement of the protein in the dry state) and plate fluorescence was measured with an excitation between 269 and 280 un and an emission of 325 to 475 mu using a Tecan Sparc plate reader in top-read emission mode.

The optimal z-position for fluorescence protein concentration quantification on the S-Trap 96-well plate using a Tecan Sparc was experimentally determined on bound protein to be 2100 μm. Protein fluorescence in both a wet and dry state was measured with an excitation between 269 and 280 nm and an emission of 325 to 475 nm using a Tecan Sparc plate reader in top-read emission mode. Protein concentrations determined via protein fluorescence were compared to BCA for limit of detection, reproducibility, and dynamic range. The optimal excitation and emission were experimentally found to be excitation at 277 and emission at 360 nm.

The optimal z-position for fluorescence protein concentration quantification on the S-Trap 96-well plate using a Tecan Sparc was experimentally determined on bound protein to be 2100 μm. Protein fluorescence in both a wet and dry state was measured with an excitation between 269 and 280 nm and an emission of 325 to 475 nm using a Tecan Sparc plate reader in top-read emission mode. Protein concentrations determined via protein fluorescence were compared to BCA for limit of detection, reproducibility, and dynamic range. In general, the optimal excitation and emission were experimentally found to be excitation at 277 and emission at 360 nm, though 410 nm afforded additional sensitivity (see below).

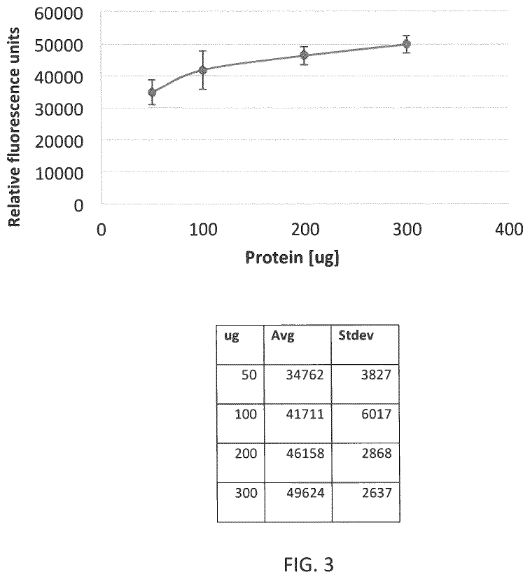

For reduced and alkylated serum at 50, 100, 200 and 300 ug loads prepared as above, placed in the digestion buffer (50 mM TEAB pH 8) which is the next step of S-Trap sample processing, the following results (FIG. 3) were obtained with excitation at 277 and emission at 360 am.

(Curves for BSA look very similar to FIG. 3, a fact easily understood by the facts that HSA human serum albumin is the most prevalent protein in serum and HSA and BSA are very similar.)

Response was reasonably linear up to ˜100 μg per well. After this, the linearity of response dropped significantly—the “hook” of this curve—an expected phenomenon as fluorescent moieties will bind deeper in the trap, which is not LV transparent, S-Traps, like other solid phases, load first at the top of their resin, where in this case proteins are captured and able to be detected, and as the load increases further inside the column, where the light cannot reach. It is noted that a transparent means of immobilizing the molecules of interest would not be subject to this limitation; see below example with glass beads.

Interestingly, in a dry state, likely due to quenching, tryptophan fluorescence was inversely correlated to the amount of protein bound up to 300 μg. Fluorescence was quenched as buffer pH became acidic, eventually reaching near-background levels.

One skilled in the art will recognize that from this single curve, it is possible for a given fluorescence to determine a protein concentration. The protein immobilization and washing system removes contaminants for fluorescence (such as, by simple example, aniline) as well as for other protein assays (like reductants for BCA, which could have been performed directly on the plate). The invention affords direct determination of protein concentration with intrinsic cleanup. In the case of protein immobilized on a surface for further processing by chemical, enzymatic or chromatographic steps, among other possibilities, this invention removes the need for protein assays, speeding sample analysis and increasing throughput.

This direct-determination invention afforded protein quantification in a significantly reduced time compared to BCA assays. No sample was lost and the sample was literally measured in place during the normal workflow of bind, wash, add digestion reagents and incubate to generate peptides. The dynamic range and sensitivity was compatible with standard bottom-up and top-down proteomics workflows. The invention successfully removed matrix contaminants prior to protein concentration determination without the need for additional steps. Such on-plate protein concentration determination lends itself directly to deployment in high-throughput clinical settings using automated fluid handlers.

### Example 2A: Equivalence to In-Solution Fluorescence

BSA solutions containing various amounts of protein were prepared in 5% SDS, 50 mM TEAB, The fluorescence of protein solutions were measured first in solution, then bound to an S-Trap plate exactly as in example 1. A line or second order polynomial were fit to the resulting fluorescence measurements (see below).

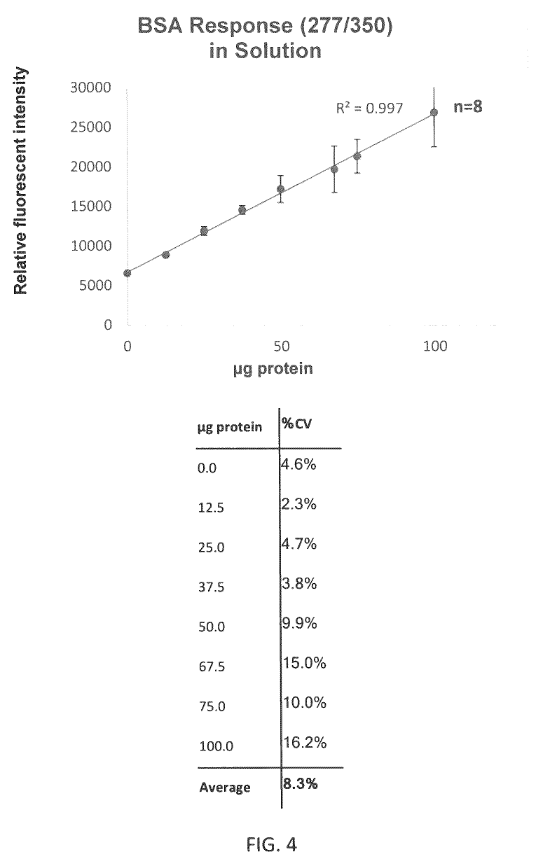

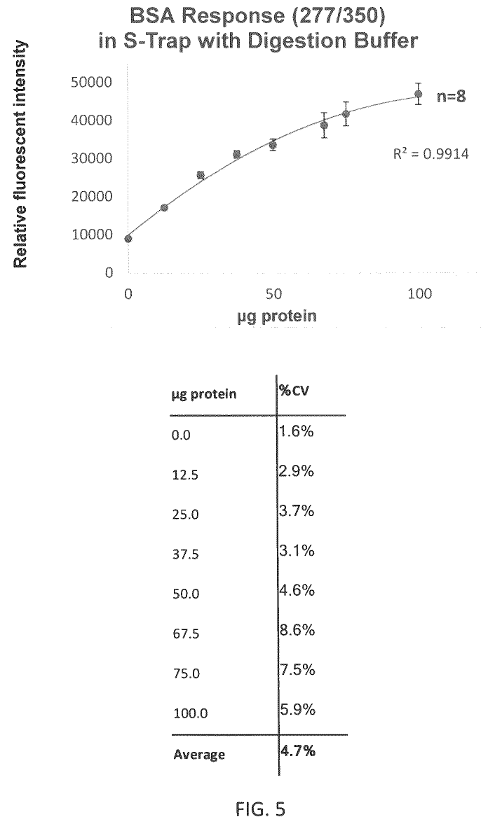

The results of this new on-plate invention compare favorably to in-solution fluorescent measurements on the same sample: both curve fits (R2) and CVs are comparable (FIG. 4 and FIG. 5).

### Example 2B: Equivalence to In-Solution BCA Assays

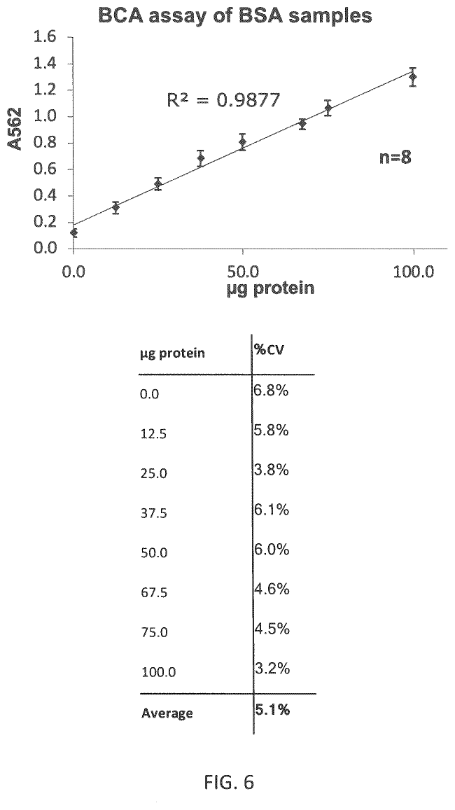

Identical aliquots of BSA in the same buffer as example 2 were assayed as per manufacturer's instructions (Pierce) and a line fit, as in the same example. Protein quantification via an on-plate fluorescence reading was of comparable accuracy to in-solution BCA of the same sample without necessitating sample loss, incubation or even further manipulation than loading onto the S-Trap 96-well plate. Again, both curve fits and CVs between a standard BCA assay and this new invention are comparable (FIGS. 5-6).

### Example 3: Sensitivity

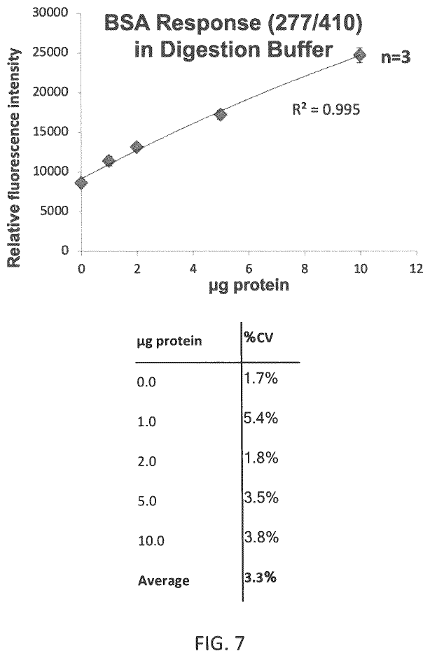

From 0 to 10 ug of BSA was solubilized as in Examples 1 and 2. Protein samples were bound to an S-Trap plate as in Example 1. Background fluorescence readings of protein-free trapping matrix decreased with increasing emission wavelength. At 277 nm excitation and 410 nm emission, it was possible to detect as little as 1 jig of protein. Such small quantities are virtually impossible to quantify by colorimetric assays yet are frequently encountered in laser capture microdissection (FIG. 7).

### Example 4: Glass Beads

FITC labeled casein (prepared in house and equivalent to Sigma) was prepared in 5% SDS, 50 mM TEAB at 1 mg/mL. 100 mg of 9-13 μm glass spheres/beads (Supelco 440345) was suspended in 1 mL of water and vortexed until suspended. Anything magnetic was removed with a strong permanent neodymium magnet. Beads were pelleted at 1000 g for 1 min and buoyant beads were discarded. After three washes beads volume was set such that the beads were at 50 ug/uL, 2 uL of this bead suspension was added to 10 ug (10 uL) of the FTIC solution. 48 uL of acetonitrile was added to the mix, the tube was vortexed for 5 sec, centrifuged for 5 min at 12,000 g, the supernatant removed taking care to not disturb the beads/pellet, 200 uL of 80% ethanol (EtOH) was gently pipetted to the beads but they were not resuspended, again centrifuged, thrice repeated removing ˜95% of the wash each time. All supernatants and washes were kept and all fractions including the now bead-bound protein, were imaged with a standard handheld laboratory UV source. All or virtually all fluorescence was on the beads. This example shows that a clear substrate can be used in this invention to achieve an extended dynamic range of quantification.

### Example 5: RNA

Yeast tRNA was purchased from Sigma and partially FITC labeled at roughly 1 out of 10 amine groups (standard FTC labeling protocols but at a slightly more neutral pH of 8.5). The tRNA was cleaned of excess FITC via multiple ethanol precipitations and multiple washes until the supernatant was clear, then it was resuspended in 50 mM TEAB at 1 mg/mL. 10 ug of this sample was bound to glass beads exactly as in Example 4 except that in place of acetonitrile, ice-cold ethanol and isopropanol were used (in separate tubes) and the 80% EtOH wash was precooled on ice. Again, all fractions were kept and monitored by UV and again all or virtually all fluorescence was on the beads.

This example could have used intrinsic fluorescence of DNA and/or RNA with excitation around 267 n and emission around 330 nm. In this case, care would need to be taken of the glass used as glass has different transparencies to UV. Also, the sample could have just as easily been applied atop a filter or chromatographic resin (e.g. ion exchange resin) to become physically immobilized, where upon it is washed with a solution known to one skilled in the art to not dissolve nucleic acids which does dissolve small molecules (e.g. 75% ethanol). The so immobilized nucleic acid can then be quantified by fluorescence, and in a column would be subject to the same “hook” effects seen in Examples 1 and 2. This example demonstrates that this invention works nucleic acids; again the examples herein are not intended to be limiting.

### Example 6: Removal of Reductant with Example of Antibody

A solution of goat IgG was prepared at 1 mg/mL in 50 mM TEAB. The reductant TCEP was added to this solution to a final concentration of 10 mM and 100 ug (100 uL) of the solution was placed in multiple wells of a standard ELISA. Control wells had exactly the same buffer, but no IgG. The plate was incubated overnight at 4 C then the wells were emptied and washed thrice with 200 uL of PBS. To this was added working BCA reagent. No color developed in the control wells, as expected because the TCEP does not bind to ELISA plates, and the wells containing now adsorbed antibody turned purple. Had a standard curve been included, this experiment would quantify the binding capacity of the ELISA plate.

### Example 7: Enzyme Immobilization

Commercial goat anti-rabbit horseradish peroxidase (HRP) was provided with a small quantity of Coomassie brilliant blue to color the solution for visualization and to one set of samples 0.25% NaN₃, an inhibitor of HRP, was added. Control beads were treated the same but used BSA in place of HRP. The sample was bound to beads as in examples 4 and 5 however the beads, after washing, were allowed to dry and were left at room temperature for two days. (As expected, all blue color was removed.) Subsequently, the beads were rehydrated in 40 uL of 3,3′,5,5′-tetramethylbenzidine (TMB) HRP substrate. The control beads did not develop any color and the blue of the HRP exposed to NaN₃. This example shows that enzyme activity can remain after immobilization of proteins, and that this activity was stable at room temperature.

### Example 8: Sample Stability

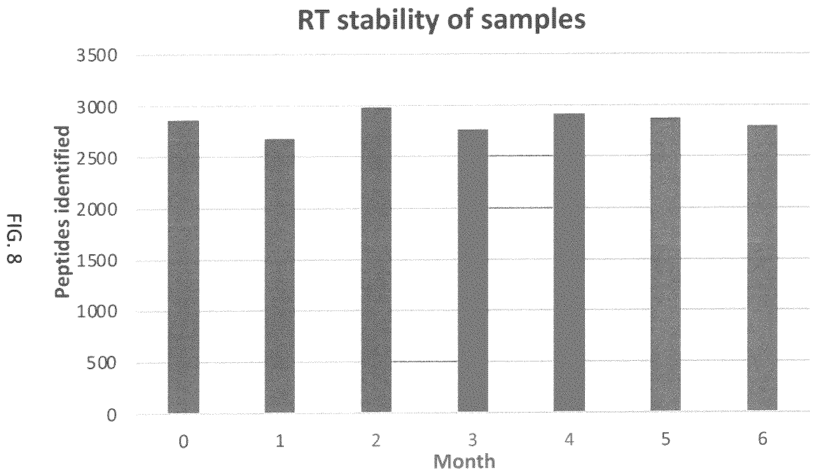

Multiple aliquots of 100 ug of serum in 5% SDS, 50 mM TEAB was reduced and alkylated as per the recommendations of the S-Trap protocol with TCEP and MMTS. 10 such aliquots were immobilized and washed per standard protocols on S-Trap mini spin columns. One column was placed at −80 C. The other columns were all left at room temperature on the benchtop, with the only additional care that they were kept in a zip-lock bag for protection from dust. Every month for 6 months one spin column was placed at −80 C such that there were 7 columns “aged” at room temperature from 0 to 6 months. These were processed as per the standard S-Trap sample processing protocol (1:10 wt:wt trypsin, 2 hrs at 47 C) and analyzed on an Agilent 6546 on a 2.1 mm column with a 60 min gradient in positive ionization mode. The number of peptides identified was as in FIG. 8.

These results indicate that the sample was stable at room temperature.

### Example 9: Sample and Protease Stability

At the same time as Example 8, 10 ug trypsin was added immediately before acidification and binding. These samples were treated identically to Example 8, except that they were simply rehydrated in 100 uL of 50 mM TEAB upon the day that all digestions of Example 8 took place. Peptide identification rates were statistically indistinguishable from Example 8, indicating that the protease and sample were stable at room temperature for at least 6 months.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method of quantifying target molecules comprising the steps of:
binding target molecules to a surface, wherein the target molecules are presented for a quantification assay;
cleaning the target molecules of contaminating reagents, wherein the target molecules remain bound to the surface;
directly quantifying the target molecules, wherein the target molecules remain bound to the surface, wherein direct quantification of the target molecules is performed by measurement of intrinsic fluorescence of the target molecules.

2. The method of claim 1, further comprising storing or transporting the target molecules at least at room temperature, wherein the target molecules remain stable while bound to the surface.

3. The method of claim 1, wherein the target molecules have a greater affinity for the surface than the affinity for the surface exhibited by the contaminating reagents.

4. The method of claim 1, wherein the direct quantification occurs by use of spectrophotometric techniques.

5. The method of claim 1, wherein each step of the method is automated.

6. The method of claim 1, wherein the surface is a C18 hydrophobic surface or optionally C4, C8, or other suitable hydrophobic surface.

7. The method of claim 1, wherein the target molecules are bound to a surface by hydrophobic or hydrophilic chromatography.

8. The method of claim 1, wherein the target molecules are bound to a surface by weak or strong ion exchange (cation or anion).

9. The method of claim 1, wherein the target molecules are bound on a surface presented on one or more selected from the group consisting of beads, membrane, packed column, monolithic column, glass beads and chromatographic beads.

10. The method of claim 1, wherein the target molecules are bound on the surface and washed of reducing reagents.

11. The method of claim 1, wherein the target molecules are bound on the surface and washed of aniline.

12. The method of claim 1, wherein the direct quantification is performed using a bicinchoninic acid assay.

13. The method of claim 1, wherein the direct quantification is performed by measuring protein fluorescence.

14. The method of claim 1, wherein the target molecules are nucleic acids.

15. The method of claim 14, wherein the target molecules are RNA.

16. The method of claim 1, wherein the target molecules are proteins.

17. The method of claim 1, wherein the intrinsic fluorescence of tryptophan is measured.

18. An apparatus for implementation of the method of claim 1, wherein the device comprises:
a protein immobilization spot;
a UV light source; and
a detector.

19. The apparatus of claim 17, wherein the apparatus comprises a 96-well plate.

20. The apparatus of claim 18, wherein the apparatus is automated.
